(19) United States
(12) Patent Application Publication
(10) Pub. No.: US 2024/0157094 A1
(43) Pub. Date: May 16, 2024

(54) WIRE DEVICE AND METHOD FOR SELECTIVELY STIFFENING A WIRE DEVICE

(71) Applicant: SoftRail Medical AG, Basel (CH)

(72) Inventors: Alexander VON WEYMARN, Frauenfeld (CH); Thomas GERIG, Burgdorf (CH)

(73) Assignee: SoftRail Medical AG, Basel (CH)

(21) Appl. No.: 18/549,730

(22) PCT Filed: Feb. 28, 2022
(86) PCT No.: PCT/EP2022/054920

§ 371 Date: Sep. 8, 2023

(30) Foreign Application Priority Data
Mar. 9, 2021 (EP) 21161564.6

(51) Int. Cl. A61M 25/09; A61M 25/00

(52) U.S. Cl. CPC A61M 25/09; A61M 25/005; A61M 2025/0915

(57) ABSTRACT

The invention relates to a wire device (100), in particular a wire device for insertion in a body lumen, comprising a stiffening portion with a pressure tube (2), a stiffening layer (3) and an outer tube (4). The stiffening layer is arranged concentrically about the pressure tube, and the outer tube is arranged concentrically outside of the stiffening layer. The outer tube is radially stiff and the stiffening layer is radially movable. Pressure can be built up inside the pressure tube such that the stiffening layer can be forced against the outer tube from the inside and the stiffening portion can be reversibly stiffened under pressure. The pressure in the pressure tube can in particular be reduced so that the stiffening portion becomes flexible again.

The invention is directed to a wire device and a method for selectively stiffening a wire device.

In minimally invasive surgical procedures, the use of a wire device is common. Such a wire device can be used, for example, to guide a catheter, especially in narrow vessels. For this purpose, a wire device should allow torque transmission.

Such a wire device can be used, among other things, to remove a thrombus. For this purpose, the thrombus can be punctured and subsequently aspirated through a catheter.

In many cases, such wire devices must be able to travel, often long distances, inside the patient's body, make turns and angles along blood vessels, and be insertable even into small blood vessels.

Furthermore, it should be possible to use the wire device in combination with tools and/or surgical treatment elements such as stents, coils or the like.

In these medical treatments, it is particularly important to be able to work as precisely and minimally invasively as possible, as the patient's life may be at risk.

Since the wire device must be elastically deformable in order to adapt to the coils of the vessel, to be steered and inserted, the wire device can very easily slip or be deflected. On the one hand, this is very dangerous because it is difficult to work precisely and the treatment can be prolonged. On the other hand, there is a risk that the patient may suffer damage or even die.

For example, an aneurysm could rupture, or calcium deposits present in the vessels could detach due to improper slippage and cause severe damage.

In many cases, it is necessary to change to a different wire device during treatment, so material consumption is very high and so are the costs.

It is therefore an object of the present invention to overcome these disadvantages of the prior art and to present a wire device with which minimally invasive surgical procedures can be performed with as little risk as possible, quickly, and costs can be kept low.

The task is solved by a wire device and a method according to the independent claims.

In particular, the problem is solved by a wire device, especially a wire device for insertion into a body passage, comprising a stiffening section with a pressure tube, a stiffening layer and an outer tube. The stiffening layer is arranged concentrically around the pressure tube, and the outer tube is arranged concentrically outside the stiffening layer. The outer tube is radially rigid and the stiffening layer is radially movable. Pressure can be built up in the pressure tube in such a way that the stiffening layer can be pressed from the inside against the outer tube and thus the stiffening section can be reversibly stiffened under pressure. In particular, the pressure in the pressure tube can be relieved so that the stiffening section is flexible again.

The wire device thus has the advantage of selective reversible stiffening.

The preferred pressure can essentially be in a range of 3-40 bar, in particular essentially at 16 bar. Other pressure ranges are also conceivable as long as sufficient stiffening is ensured.

Radially rigid in the sense of the invention means that the radially rigid layer can be expanded only minimally by pressure in the radial direction, so that pressure can be applied at all.

Radially movable means that the layer is not radially rigid.

Radially rigid does not exclude that the layer is flexible in other directions.

Preferably, the pressure can be applied, checked and released by an external device, which may include further protective measures, such as shutdown in the event of a pressure drop due to a leak. Alternatively, the pressure can also be applied manually.

At this point, it should be noted that the pressure difference to the human body must be taken into account. Therefore, the wire device is designed in such a way that a high pressure can be supplied and discharged. Preferably, a pressure above atmospheric pressure is used and not a vacuum or negative pressure. This has the advantage that the pressure difference is much higher, since the negative pressure is limited to a maximum value of 1 bar.

The formation of a stiffening section of the stiffening section facilitates the fixation of the wire device in the desired location and allows for a more precise procedure that increases patient safety, is more feasible to perform, and ensures faster work.

A wire device is preferably designed such that a device, in particular a catheter, can be slid onto it. The wire is preferably arranged centrally in such a catheter and is completely or at least partially enclosed by the catheter.

Thus, the wire device is preferably suitable for guiding a catheter in vessels by allowing the wire device to perform bends in the vessels.

In the stiffened state, the pressure can also be reduced again and the stiffening can be reversed.

Individual sections, such as a front section, a rear section, and/or a middle section of the wire device may be designed to be stiffened. Thus, the stiffening section of the wire device may comprise the entire wire device or only a partial section.

Provided that only a partial section of the wire device is formed by the stiffening section, the rest of the wire device is preferably not designed to be stiffenable.

Inside the wire device is an interior space surrounded by the pressure tube. The interior space is preferably not reduced in proper use by kinks or pressure on the wire device.

The interior space preferably has an opening to the distal end of the wire device and is preferably closed at the proximal end of the wire device, which is inserted into the vessels. The interior of the pressure tube may also be closed before the proximal end.

In this context, the formation of multiple stiffening sections in a wire device is conceivable. Preferably, the pressure tube extends at least from the distal end of the tubing apparatus to the proximal end of the last stiffening section.

It is important for the function of the wire device that the outer tube is radially rigid. This radial stiffness means that there is no or only slight expansion of the outer tube. This ensures that the outer tube allows pressure to build up and that, unlike the stiffening layer, the outer tube is essentially not radially movable.

Nevertheless, high flexibility and bendability of the wire device in the unstiffened state is possible to allow the wire device to complete turns along the vessels and branches of the vessels.

The radial mobility of the stiffening layer, on the other hand, is important so that the stiffening section can be stiffened by pressure. Stiffening is possible by form fit and/or force fit if the stiffening layer can be pressed against the outer tube from the inside by the built-up pressure.

The pressure tube of the wire device can be expandable, in particular elastic and/or unfoldable.

The pressure tube can be subjected to pressure, thus pressing the stiffening layer against the outer tube and stiffening the stiffening section of the wire device. This arrangement offers the advantage of enabling simple selective stiffening in a miniaturized design.

It should be noted that this arrangement is advantageous for potential leaks in the pressure tube. The safety of the patient would be guaranteed despite a leak, as the pressure is built up in the pressure tube. Thus, a leak would have to occur in the pressure tube and in the outer tube for the pressure to escape via the outer tube.

Preferably, the outer tube is designed to be pressure-tight.

Furthermore, this allows pressure to be applied to the stiffening layer without the need for the stiffening layer to have a pressure-tight layer. This can reduce the resistance to radial movement.

A stiffening layer of spirally arranged elements is also conceivable. In addition, a stiffening layer is conceivable which forms a closure with a complementarily formed inner surface of the outer tube for stiffening the wire device.

Preferably, however, the stiffening layer is not monolithic.

This preferred design of the wire device allows for a more flexible design of the stiffening layer, as it may have a non-closed, perforated, grooved, spiral, mesh, interwoven and/or net structure, construction and/or surface.

This can have an advantageous effect on the stiffening of the stiffening section, as the stiffening section can thus be stiffened more easily during severe bending.

The pressure tube can expand when subjected to pressure, and preferably comprises elastically deformable material, and/or a foldable material, such as a plastic.

Nylon, polyethylene terephthalate and/or polyether block amide are particularly suitable for an expandable and retractable pressure tube.

In particular, the pressure tube advantageously comprises stretchable elastomers or other thermoplastic plastic material, such as silicone and/or thermoplastic elastomers and/or foldable plastics. These materials have a high biocompatibility, are non-toxic, are inexpensive, and are preferably very elastically deformable.

Particularly advantageous is a pressure tube that can have a collapsed state without the action of additional pressure and can have an unfolded state when pressure is applied. Such an unfoldable pressure tube ensures a fixed maximum and minimum size when applying proper pressures and can provide stiffening with maximized tightness.

The stiffening layer may include at least two stabilizing elements. The orientation of the stabilizing elements may have at least a partial vector in the longitudinal direction of the wire device. Preferably, the stabilizing elements are movable relative to each other.

The individual stabilizing elements can be movable, in particular displaceable, relative to one another in the unstiffened state. Preferably, the stabilizing elements have little tensile elasticity and preferably also little compressive elasticity in the longitudinal direction.

The stabilizing elements of the stiffening layer are preferably formed to a higher degree along the longitudinal direction, so that the angle of the stabilizing elements between the longitudinal axis of the wire device and the stabilizing element is formed in a range between −45° and 45°, in particular between −30° and 30°. It is also possible to provide different orientations, i.e. two or more different angles to the longitudinal axis of the stabilizing elements with respect to the longitudinal axis. This has the advantage that the bendability is only slightly restricted, but the form fit is strengthened during stiffening in the longitudinal direction between the stiffening layer and the outer tube and preferably the pressure tube.

The stabilizing elements are preferably shaped to have high friction with the outer tube due to high pressure in the direction of the outer tube and can lead to stiffening of the wire device.

The stiffening layer and/or stabilizing elements preferably comprise metals and/or plastics.

Stainless steel or polyamide, for example, are particularly suitable.

This cohesion between the individual stabilizing elements of the stiffening layer can be formed by mechanical adhesion, such as positive interlocking in recesses, and/or by specific adhesion.

In this context, it would also be conceivable to use stabilizing elements of the outer tube or use additional stabilizing elements on the inside of the outer tube, which can contribute to stiffening, for example by means of a form fit and/or force fit.

The stabilizing elements of the wire device are preferably made of metal and/or plastic.

In particular, a plurality of stabilizing elements is preferably interlaced to form a braided structure.

Metals and plastics have very good elastic properties and are usually inexpensive. Furthermore, good elasticity of the material is important so that when the applied force is removed by the pressure, the stabilizing elements can return to their original shape.

This ensures that the wire device can be stiffened, but can also become elastically deformable again when the pressure is reduced.

It is also advantageous if there is no strong elastic hysteresis of the material, i.e. no deformation can remain after the deflecting forces have been removed. Elastic hysteresis is counteracted by the structure of a braided structure, use of an elastic material and/or non-plastic deformation.

The elasticity of the stiffening layer can also be achieved by the type of stiffening layer instead of or in addition to the material elasticity. A relatively loose braided structure can achieve a certain elastic radial deformation by applying a pressure even if the material of the braided structure is not or hardly elastic per se.

The use of stabilizing elements made of metal and/or plastic also permits cost-effective manufacture. A braided structure can be manufactured very quickly by machine, thus reducing the cost of the invention.

A particularly advantageous design results for the angles described above between −45° and 45°, in particular between −30° and 30°, in combination with a braided structure.

Nevertheless, other manufacturing options for creating an advantageous structure of the stabilizing elements, such as laser cutting, would also be conceivable.

The outer tube of the wire device is preferably pressure-tight and in particular made of a plastic and/or metal.

The outer tube preferably has a wall thickness in a range of 0.01 to 0.8 mm, in particular 0.07 mm, and may comprise materials such as stainless steel, platinum, iridium, barium, tungsten and/or plastics, in particular thermoplastics such as silicones, polyetheretherketone, fluoropolymers, polyvinylchloride and/or polyurethane.

When choosing the material, care must be taken to ensure that both the mechanical properties, in terms of flexibility, the surface structure, in terms of thrombogenicity, and the chemistry of the surface, in terms of coatability and charge, is advantageous. The above materials exhibit these advantageous properties and are thus suitable for use in the wire device.

Preferably, the outer tube is also reinforced so that the cross-section is not so greatly enlarged and/or compressed under pressure. Preferably, the wire device has a high resistance to pressure. For the reinforcement, the outer tube can be stabilized by spirals, meshes, a wire or a braided structure with transverse orientation. The reinforcement structure is preferably optimized for a ratio of resistance of radial expansion and longitudinal expansion, for example, by adjusting the thickness of the reinforcement and the angle to the wire device.

The choice of these materials and an advantageous reinforcement for the outer tube ensures that the wire device is elastically deformable but cannot be compressed during proper use. In the longitudinal direction, the outer tube should also preferably not be elastically deformable, or hardly so.

The wire device may include a torsionally rigid structure, preferably a hypotube, helical and/or twisted structure.

Preferably, a rotation of the wire device in the region of the distal end of the wire device may cause an analogous rotation in the region of the proximal end of the wire device.

Thus, the wire device can be rotated in the vessels from the distal end, which is preferably located outside the vessels.

The transmission of torque is preferably substantially 1:1, so the wire device is preferably configured such that rotation in at least one direction of the wire device at the distal end through an angle results in substantially the same rotation through the angle at the proximal end.

A push-on device, in particular preferably a catheter and/or dilator, can thus be controllable by the structure provided by the wire device.

The push-on device, in particular the dilator, preferably has a lower bending stiffness than the guide wire in the stiffened state. The push-on device can also be referred to as an adapter.

Preferably, the wire device can form a specific orientation by stiffening the stiffening section.

The slide-on device can be slid along this orientation.

Preferably, the torsionally rigid structure of the wire device is configured to transmit torque about the longitudinal axis of the wire device along the wire device in at least one direction.

A directional transfer of torque of the wire device may be formed by a particular winding, armoring, cuts, and/or removal of material.

In this context, it is conceivable that the rotation of the wire device for alignment in the vessels can only be performed in one direction.

Thus, the structure, which is torsionally rigid in one direction, can preferably be designed with lower material consumption and/or thinner layers.

The wire device can also be designed to be torsionally rigid in both directions of rotation about the longitudinal axis of the wire device.

The torsionally rigid structure can be arranged in the stiffening layer and/or the outer tube and/or the pressure tube.

It would also be conceivable to use the torsionally rigid structure as an additional layer of the wire device.

The torsionally rigid structure can also have good shape recovery properties.

Preferably, the wire device is designed to be stiff against buckling and/or compression, especially when rotated about the longitudinal axis.

The torsionally rigid structure may include metals and/or plastics. In particular, the torsionally rigid structure may include stainless steel, nickel titanium, tungsten, a fluoropolymer layer, general purpose resins, polyamides, and polyetheretherketones.

The torsionally rigid structure may be formed by a tubular structure. This tubular structure can also have sections and/or distant surface segments, in particular at regular and/or alternating intervals.

The torsionally rigid structure can be formed by a hypotube. The hypotube can be manufactured using process technologies such as laser cutting or core wire welding.

The tubular structure may be modified by laser cutting, especially in the circumferential direction.

The torsionally rigid structure may be formed by a twisted and/or helical structure. In this context, helical structures that are torsionally rigid in one or both directions of rotation about the longitudinal axis of the wire device are conceivable.

In particular, a helical and/or at least partially helical arrangement of the cuts and/or the remote surface segments of the torsionally rigid structure is conceivable.

The torsionally rigid structure may have multiple layers with different orientations that provide anisotropic force application of the wire device.

The wire device can comprise a fluid coupling through which a fluid can be introduced into the pressure tube. In particular, a cross-section through the fluid coupling has at most the same extent as a cross-section of the stiffening section.

The fluid coupling is preferably designed to be able to build up and/or release a pressure by connection to a pressure device.

The fluid coupling is preferably designed such that a fluid can be introduced into the pressure tube by a pressure device.

Preferably, the fluid can be removed from the fluid coupling again by the pressure device.

The fluid coupling preferably has a smaller, or only slightly larger, extension in cross-section than the stiffening section.

Preferably, this allows a push-on device, especially preferably a catheter and/or dilator, to be slid over the wire device.

The fluid coupling can have sealing devices, in particular plastic seals.

As a specific embodiment, the use of a check valve and/or a removable valve as a fluid coupling would be conceivable in this context.

The closing element of a check valve can preferably be moved from a closed to an open state by a mechanism.

Thus, preferably, the flow of a fluid may be possible in the open state and may be closed in at least one direction in the closed state due to the pressure of the fluid.

The fluid coupling may be connectable attachable to the wire device.

In particular, the use of a push-on and/or screw-on valve as a fluid coupling is conceivable. When attaching such a fluid coupling, a fastening device can also be provided, in particular clamps and/or clips.

The fluid can be a liquid, for example water, salt water or even a gas. Thus, the introduction of a fluid into the pressure tube allows the stiffening section to be stiffened. However, it is preferable to use a fluid, as this is less dangerous to humans when escaping than a gas, especially air.

The wire device may preferably comprise, at least in part, an X-ray visible material, in particular metal.

In minimally invasive operations with wire devices, the position of the wire device, in particular the inserted proximal end of the wire device, may not be visible to the naked eye.

Accordingly, an imaging technique must preferably be used to visualize the inserted wire device. Preferably, an X-ray method such as fluoroscopy is used so that real-time imaging of the inserted wire device can preferably be ensured.

Since the attenuation of X-rays is given by the mass attenuation coefficient, a material with elements of a high atomic number should be chosen.

Since plastic often consists mainly of carbon compounds that absorb X-rays poorly, the wire device should instead contain visible material such as modified plastic or metals. Even small amounts of iron, for example, can ensure the visibility of the wire device.

The proximal end of the wire device may have a tip. The tip may seal the proximal end of the wire device in a pressure-tight manner. The length of the tip is preferably in a range of 0.2 mm to 15 cm.

According to the invention, the proximal end of the wire device with the tip is the part that is inserted first into the patient's vessel.

The length of the tip in this context refers to the length of the tip in the direction of the longitudinal axis of the wire device.

The tip preferably seals the pressure tube of the wire device pressure-tight.

The tip can be arranged flush with the outer tube and also connected to the outer tube in a pressure-tight manner.

The tip is preferably located directly adjacent to the stiffening section.

In one embodiment, the tip has essentially the same cross-sectional area as the stiffening section. However, depending on the intended use, larger or smaller cross-sectional areas than those of the stiffening section are also conceivable.

The tip, or a region of the tip, may have a different elasticity, than the remaining regions of the wire device and/or stiffening section.

In particular, the other elasticity of the tip can be formed by other reinforcement, arrangement of the stiffening elements, and/or formation of the torsionally rigid structure.

The tip may also have varying elasticity in different areas of the tip.

The elasticity of the tip can be designed to be higher or lower than the elasticity of the stiffening section, depending on the intended use.

The length of the tip is preferably as short as possible, i.e. less than 10% of the length of the stiffener section.

Thus, preferably, as small an area as possible at the proximal end of the wire device is not part of the stiffening section.

In one conceivable embodiment, the tip is also at least partially stiffenable.

The tip of the wire device may be at least partially conical, rounded, spherical, elliptical, circular cylindrical, and/or stepped cylindrical.

This tip design offers the advantage of minimizing the risk of injury when using the wire device.

When inserting the wire device into a patient, such a tip of the wire device causes less or no injury and pain.

In addition, such a tip minimizes injury to vessels and potential loosening of deposits or clots.

A hydrophilic or hydrophobic coating may be disposed on the outside of the outer tube of the wire device.

The coating of the outer tube can, among other things, improve the sliding behavior and minimize friction. On the other hand, biocompatibility must be ensured. The risk of infection should be kept low, and hemocompatibility, antithrombogenicity and/or low particle release should be optimized. Coatings using polymers are particularly suitable for this purpose.

In the coating processes, the surface is preferably activated beforehand to be able to accept coatings. This can be done, for example, by plasma activation, in which an electron plasma causes open bonding sites to form on the surface, which are occupied by polar hydroxy groups. This creates a hydrophilic surface and the polymer coating can adhere better.

The surface to be coated can be made hydrophilic or hydrophobic, depending on the precursor monomers, for example, by plasma polymerization in a vacuum by feeding gas with precursor monomers deposited on the surface. Other coating processes such as dip coating, spray coating, reel-to-reel coating are also conceivable.

In a final step, the molecules of the coating can be further crosslinked by applying thermal energy or UV radiation.

Polytetrafluoroethylene is suitable as a hydrophobic coating for the outer tube, since the molecule is nonpolar due to its symmetrical structure, has very low surface tension, and exhibits low cohesion and adhesion forces to other substances.

Other polyfluoroethylene, hexafluoropropene and/or polydimethylsiloxane are also conceivable as hydrophobic coatings, with polydimethylsiloxane even having an advantageous antithrombogenic effect.

Hydrophilic polymers, on the other hand, are coatings that can form hydrogen bonds so that a film of water can be easily bound to the surface. Hydrophilic coatings can consist of only one hydrophilic polymer, or comprise a combination of hydrophilic polymers.

Advantageous hydrophilic coatings that can be imagined include hydrogels or polyvinylpyrrolidone, the properties of which can be further adapted by adding vinylpyrrolidone copolymers.

The stiffening section of the wire device may form part or all of the wire device.

The length and area of the stiffening section can advantageously be easily adapted to the particular problems. A length of the stiffening layer in a range of 2 cm to 100 cm is particularly advantageous, especially preferably around 30 cm. The stiffening section is preferably formed up to the proximal end and/or the tip.

Thus, the stiffening section can occupy only the area needed to hold the wire device in place in one area. The rest of the wire device can benefit from the advantages of not having a stiffening layer, such as increased elasticity and/or reduced layer thickness. Thus, costs can also be reduced while still ensuring complete functionality.

The total length of the wire device is preferably between 100 cm and 400 cm, in particular 160 cm and 260 cm, depending on the desired use.

The outer tube of the wire device may have an outer diameter of no more than 0.035 inch, particularly 0.018 inch, more preferably 0.014 inch or 0.012 inch.

A small outer diameter is often advantageous for minimally invasive procedures and also allows neurological interventions.

A small outer diameter is necessary in order to find space in narrow vessels and/or to be able to complete turns of up to about 360° with the wire device in vessels. Therefore, it is a particularly advantageous feature of the invention that the wire device is fully functional even with a small outer diameter and can be manufactured at low cost.

The pressure tube of the wire device and the stiffening layer can be connected to each other.

The pressure tube and the stiffening layer can be designed to move radially together.

Preferably, pressure can be built up in the pressure tube in such a way that the pressure tube and the stiffening layer can be pressed together from the inside against the outer tube.

The wire device can be designed as a guide wire and/or a shaped wire.

The wire device as guide wire allows to preset a curvature for push-on devices.

For this purpose, the wire device is preferably bent in the flexible state and stiffened by pressure with incoming fluid.

The push-on device can be pushed along the curvature specified by the stiffened guide wire.

The wire device as a shaped wire, on the other hand, can be designed so that the proximal end of the wire device can provide a bend to a push-on device in the vessels.

It would be conceivable for the wire device to provide a shape as a shaped wire to a push-on device, in particular preferably a catheter and/or a dilator, preferably in the region of the proximal end of the push-on device.

An embodiment is conceivable wherein the wire device can and/or does form a bend within the slide-on devices.

For this purpose, the shaped wire can preferably be bent and/or flexed depending on the intended use, and can be inserted into the wire device.

This has the advantage that the slide-on device can be made less rigid to accommodate the wire device.

The push-on device may also assume the predetermined shape in a region only when the wire device is stiffened and/or may be pushable along this shape.

The wire device as shaped wire and/or guide wire thus preferably does not have to be removed from the push-on device and/or replaced.

The wire device may preferably be more resilient in the unstiffened state than the push-on device, or a portion of the push-on device.

The wire device, when stiffened, may be less resilient than the push-on device, or a portion of the push-on device.

Thus, the wire device can preferably be removed and/or inserted from a push-on device with substantially no deformation.

In particular, the wire device as shaped wire and/or guide wire has bends for guiding and/or is designed to be deformable into bends.

Preferably, a bend of the shaped wire can be adjusted so that the shaped wire can be used flexibly.

The wire device can be stiffened once the wire device is positioned at a target vessel.

The wire device can also be stiffened when appropriate to complete a specific path within the vessels.

This provides safe access for the actual treatment of the target vessel.

In this context, it would be conceivable for the wire device to be designed as a shaped wire that can be stiffened in a predetermined shape.

A needle can be used to create access into an access vessel so that a wire device can be inserted into a target vessel. The needle can also be removed after insertion of the wire device.

The problem is further solved by a method for selectively stiffening a wire device as previously described.

By introducing a fluid into the pressure tube, the wire device can be stiffened by the stiffening layer and the outer tube.

Selective stiffening of the wire device provides both the flexibility needed for insertion into the vessels and the selective stability of a stiffened wire device.

Time of use is also minimized, as a prior art wire device can often be deflected from its point of use, for example, by blood flow, manipulation in the wire device, or slippage of the wire device.

Furthermore, for safety reasons, a fluid should preferably be selected in such a way that, even in the event of severe damage to the wire device, no damage to health can occur. In this context, a device for monitoring the pressure through the introduced fluid would also be conceivable.

By reducing the pressure in the interior of the pressure tube, the wire device can preferably become movable again by no longer pressing the stiffening layer against the outer tube.

Preferably, the wire device is in the elastic state when reduced to substantially atmospheric pressure and pressure in the blood vessels of a human.

It is particularly important that this stiffening can be selectively activated and deactivated.

Thus, the wire device can be inserted flexibly.

At the target site, the wire device can be stiffened for the minimally invasive surgical procedure, according to individual anatomy, and then made elastic again for removal.

Thus, preferably possible damage to health can be avoided.

Possible damage, such as strokes, can be caused by the loosening of debris or thrombi, as well as inserted tools and/or devices and/or small parts.

An at least partially stiffened wire device minimizes these risks and enables more precise work.

It is therefore of particular importance to avoid this potential damage and a major advantage of the invention is that selective stiffening of the wire device can be achieved as required by introducing a fluid into the gap in the pressure tube.

It is also advantageous that the pressure only needs to be built up to stiffen the wire device and released to make it elastic, as this ensures that the wire device can be released even if the pressure device fails.

If pressure had to be built up to make them elastic, a stiffened wire device would be in the vessels of a human being in the case of a leaking wire device. This would not be removable, or only with great difficulty, and/or at great risk to the human being.

In the following, embodiments of the invention are described in detail with reference signs. Hereby shows:

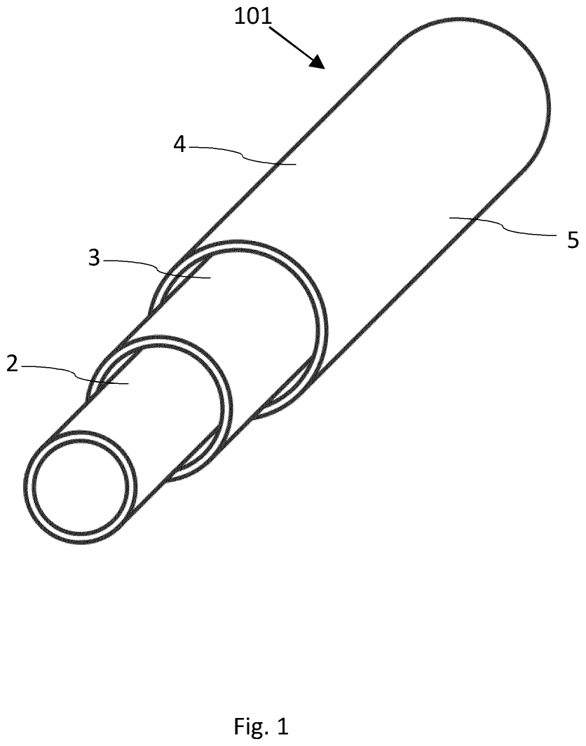

FIG. 1: A stiffening section of the wire device,

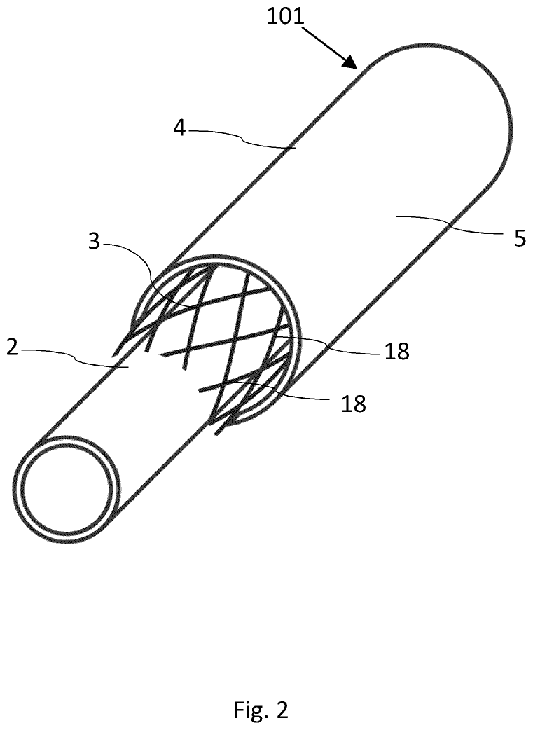

FIG. 2: An embodiment of the stiffening section,

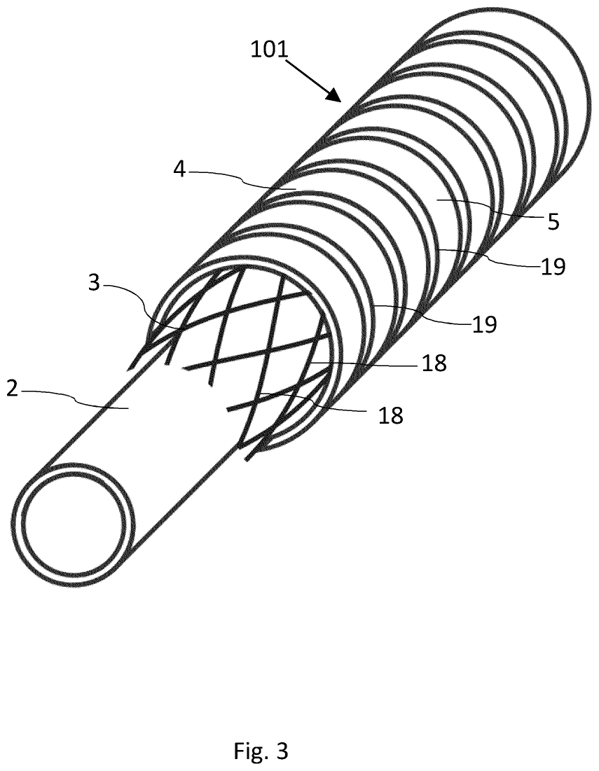

FIG. 3: An embodiment of the stiffening section,

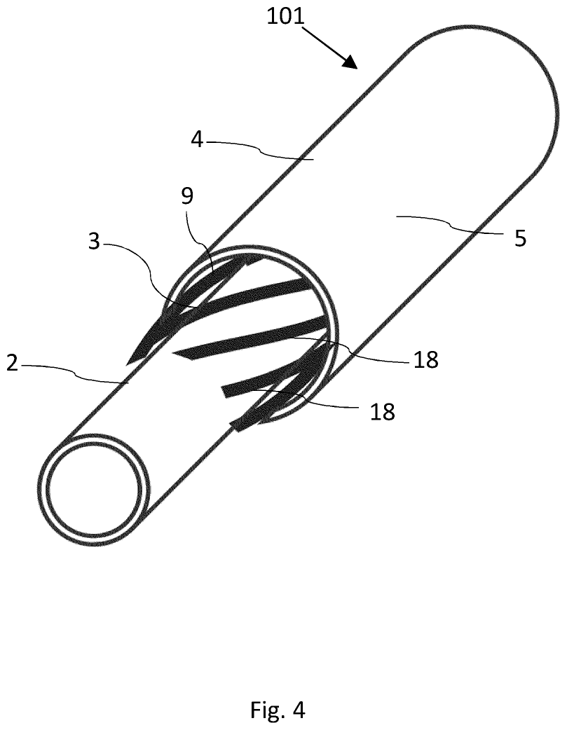

FIG. 4: An embodiment of the stiffening section,

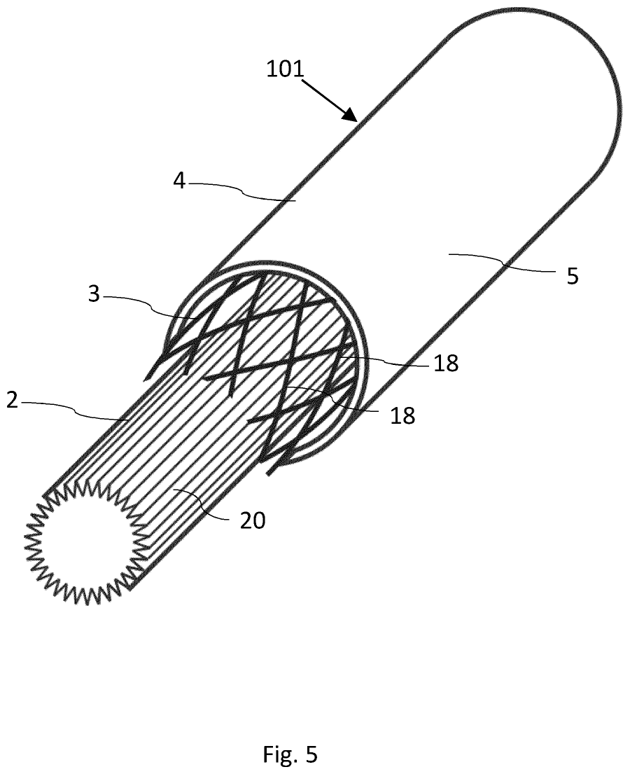

FIG. 5: An embodiment of the stiffening section,

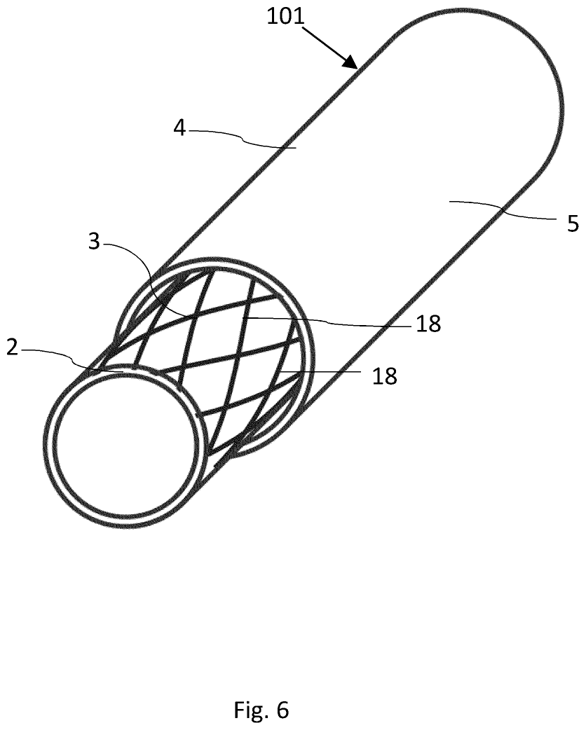

FIG. 6: An embodiment of the stiffening section,

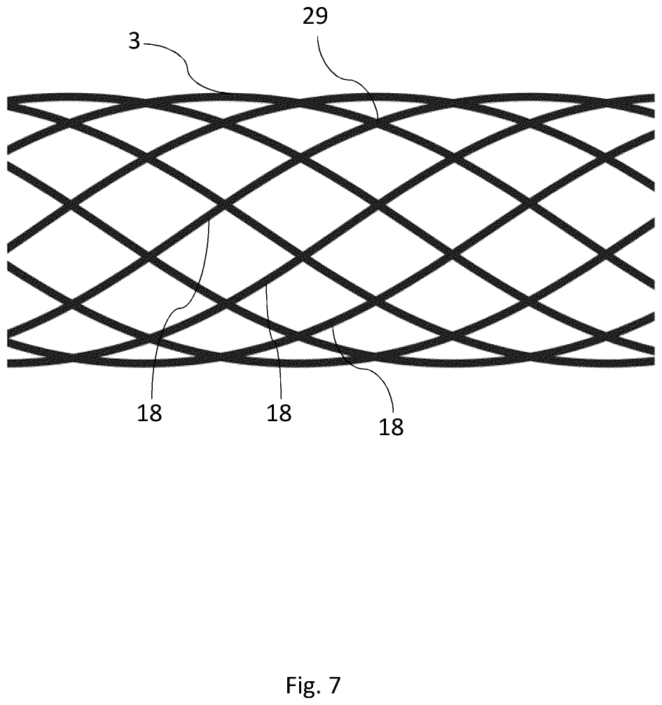

FIG. 7: A structure of the stiffening layer,

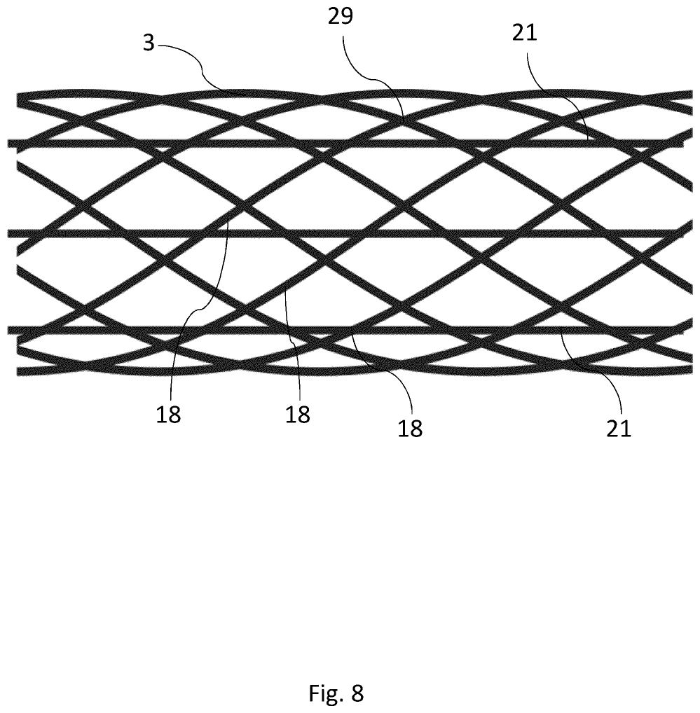

FIG. 8: An alternative structure of the stiffening layer,

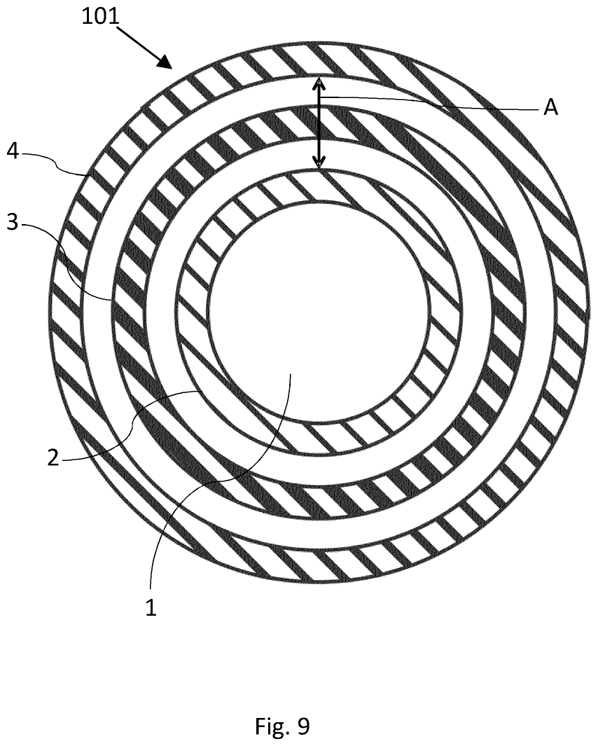

FIG. 9: A cross-section of the stiffening section of the wire device,

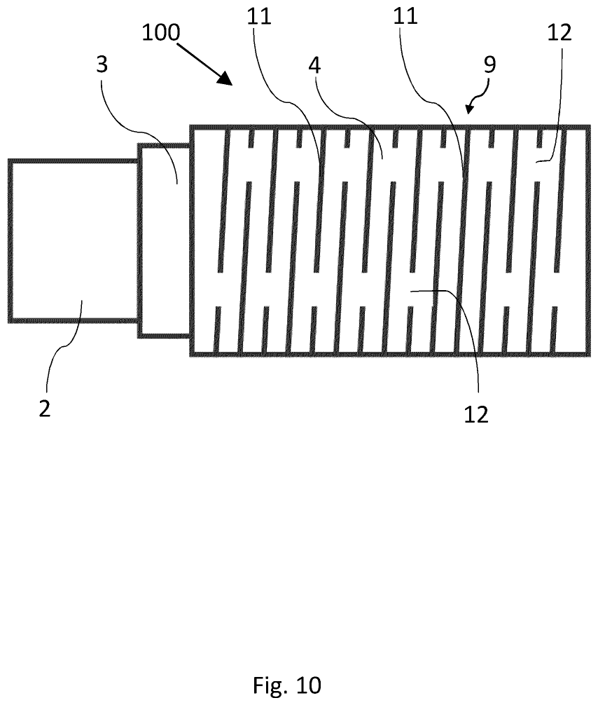

FIG. 10: A wire device with a torsionally rigid structure in profile,

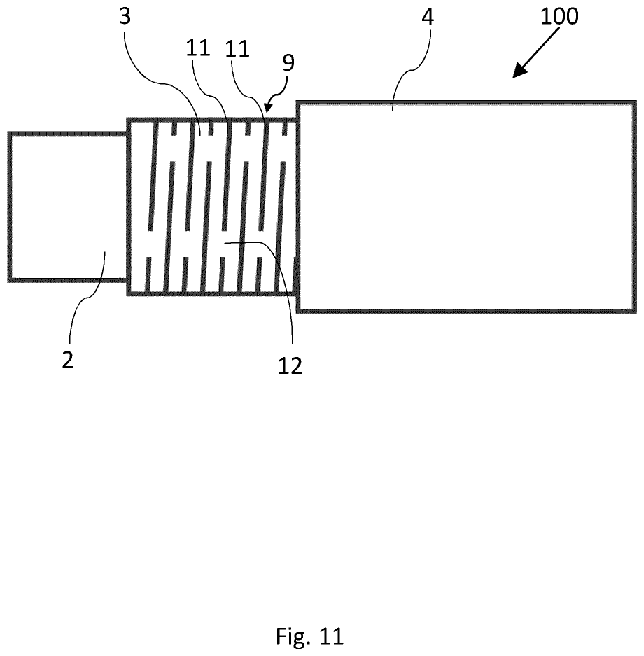

FIG. 11: A wire device with a torsionally rigid structure in profile,

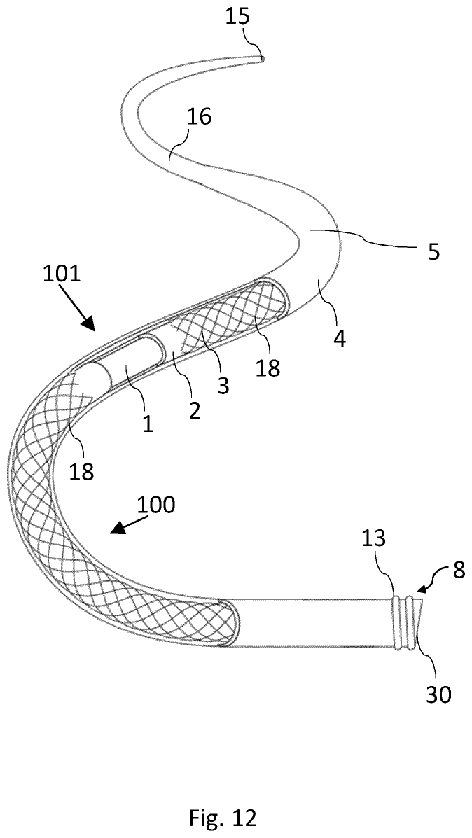

FIG. 12: A wire device having a stiffening section with partially removed layers,

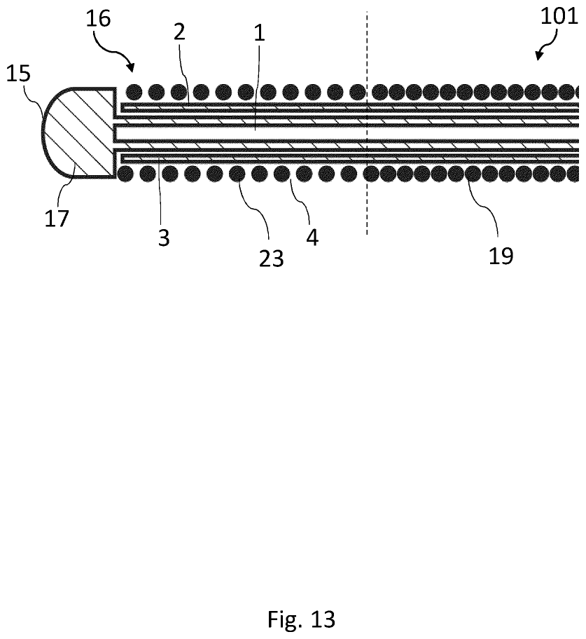

FIG. 13: A longitudinal section of a stiffening section of the wire device with a tip,

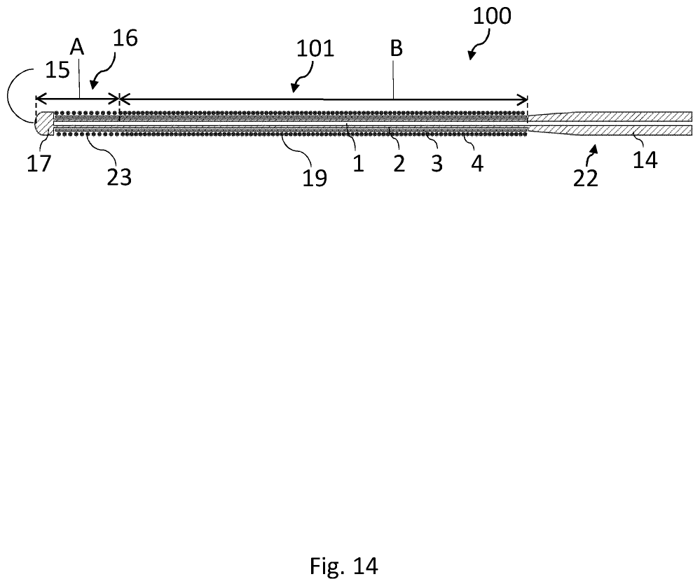

FIG. 14: A longitudinal section of a wire device having a stiffening section and a tip,

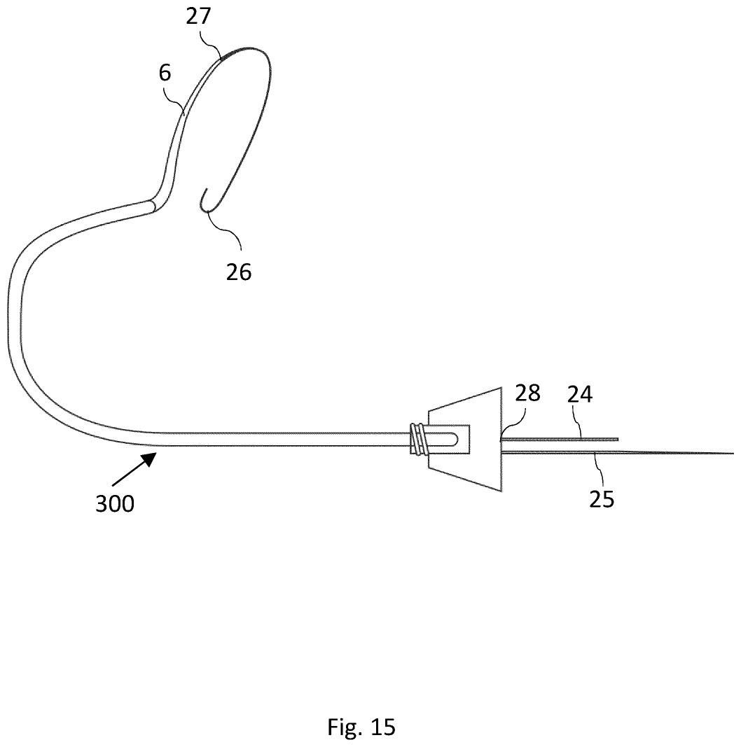

FIG. 15: An embodiment of a wire device as a shaped wire and a wire device as a guide wire.

FIG. 1 shows an embodiment of a stiffening section 101 of the wire device 100 having three layers of tubing. The wire device 100 is shown in open sections of each layer for better visibility. From the outside to the inside, there is an outer tube 4, a stiffening layer 3, and a pressure tube 2.

The pressure tube 2 is filled with an isotonic sodium chloride solution at a pressure of 16 bar for stiffening. Thus, the stiffening layer 3 presses against the outer tube 4 by moving radially outward. When the pressure is removed, the stiffening layer 3 is also moved radially inwards again and the stiffening of the stiffening section 101 decreases.

In this embodiment, the pressure tube 2 is made of thermoplastics and can thus be expanded and elastically deformed radially by the introduction of an isotonic sodium chloride solution.

The stiffening layer 3 can also be moved radially inwards again when the applied pressure is removed. This ensures that the wire device 100 can always be removed, especially when the pressure can no longer be built up.

In its basic state, the wire device 100 is designed to be movable without pressure, so that there is no danger from irreversible stiffening of the wire device 100 in the event of a defect.

In this case, the stiffening layer 3 is formed from a loose braided structure of stainless steel and/or plastic, which can be moved against each other and which runs in the longitudinal direction to form a partial vector. Thus, the stiffening layer 3 can easily expand and a strong friction with the outer tube 4 can be established.

In this embodiment, the outer tube 4 comprises polysiloxanes and a stainless steel spiral, wherein the stainless steel spiral is helically oriented along the longitudinal axis of the wire device 100, is embedded in the polysiloxane and is completely enclosed. In this context, however, the alternative use of polyurethane for the outer tube 4 would also be conceivable.

The outer tube 4 is also hydrophilic due to a coating 5 with polyvinylpyrrolidone. Thus, insertion into a body passage is easier and can be performed atraumatically. Furthermore, the coating 5 increases the sliding properties of the wire device 100 within the vessels.

FIGS. 2-5 show explicit embodiments alternatively to FIG. 1. For analogous reference signs, functions and embodiments of the invention that are not explicitly mentioned, reference is made to FIG. 1.

FIG. 2 shows the stiffening section 101 of the wire device 100, which shows a stiffening layer 3 with a braided structure made of stainless steel, the individual stabilizing elements 18 of the stiffening layer 3 being manufactured so as to be displaceable relative to one another. The stiffening layer 3 can be moved radially outwards by the pressure tube 2 and can stiffen the stiffening section 101 by friction with the outer tube 4. However, other materials for the stabilizing elements 18, such as polyamide, are also conceivable.

FIG. 3 shows an embodiment of the stiffening section 101 of the wire device 100 analogous to FIG. 2.

The wire device 100 has a braided structure of stainless steel of the stiffening layer 3 with stabilizing elements 18. In addition, the outer tube 4 has a reinforcement 19 made of stainless steel.

The reinforcement 19 is formed as concentrically as possible around the longitudinal axis of the wire device 100, so that the wire device 100 continues to be formed as flexibly as possible with respect to bending perpendicular to the longitudinal axis of the wire device when it is not in the stiffened state. In this embodiment, the reinforcement 19 is implemented by rings, but a spiral-shaped reinforcement 19 and a mesh-shaped reinforcement 19 are also conceivable.

FIG. 4 shows an embodiment of the stiffening section 101 of the wire device 100.

The stabilizing elements 18 are arranged along exactly one helical orientation around the pressure tube 2.

Such helical alignment of the stabilizing elements 18 forms a torsionally rigid structure 9.

The torsionally rigid structure 9 of the stiffening layer 3 can thus transmit torque along the entire stiffening section 101 and/or wire device 100 (not fully shown in FIG. 4) in a direction of rotation about the longitudinal axis of the stiffening section 101.

FIG. 5 shows an embodiment of the stiffening section 101 of the wire device 100, wherein the pressure tube 2 can press the stiffening layer 3 and the stabilizing elements 18 radially outward against the outer tube 4 by unfolding a folded structure 20. The maximum outer diameter of the pressure tube 2 after unfolding is larger than the inner diameter of the outer tube 4, so that the stiffening layer 3 can be pressed against the outer tube 4. Thus, the expansion of the pressure tube 2 does not change even after repeated use, since there is no or essentially no elastic deformation. The folded structure 20 of the pressure tube 2 unfolds as soon as pressure is applied with the pressure tube 2 by introducing the salt solution, and folds back in when the pressure is removed. This also has the advantage that elastic hysteresis is minimized by the folded structure 20.

Thus, it minimizes the persistence of deformation after the deflecting force is removed. Thus, the safety, as well as the durability of the pressure tube 2 of the wire device 100 is ensured.

FIG. 6 shows an embodiment of the stiffening section 101 of the wire device 100, wherein the pressure tube 2 is directly adjacent to the stiffening layer 3 and is connected to the stiffening layer 3. The pressure tube 2 and the stiffening layer 3 form a pressure-tight tube with stabilizing elements 18. The pressure tube 2 is thus radially movable together with the stiffening layer 3 and can be pressed radially outward against the outer tube 4 by an applied pressure together with the stiffening layer 3 to stiffen the stiffening section 101.

FIG. 7 shows an embodiment of the stiffening layer 3 with stabilizing elements 18, wherein the braided structure formed is not rigidly connected to one another at the interfaces 29, but is designed to be freely movable relative to one another in order to ensure radial deformability. In this embodiment example, the stabilizing elements 18 are formed at an angle of substantially 30° to the longitudinal axis of the wire device 100.

FIG. 8 shows another embodiment of the stiffening layer 3 with stabilizing elements 18 analogous to FIG. 7, with additional longitudinal stabilizing elements 21 essentially parallel to the longitudinal axis being shown, which additionally increase friction when pressed radially against the outer tube 4.

FIG. 9 shows a cross-section of the stiffening section 101 of the wire device 100 with a pressure tube 2, a stiffening layer 3 and an outer tube 4. From the outside to the inside, an outer tube 4, a stiffening layer 3, a pressure tube 2 and an inner space 1 can be seen. In addition, the area A between the pressure tube 2 and the outer tube 4, between which the pressure can cause radial movement, has been marked. Due to the applied pressure in the pressure tube 2 by inflowing fluid, a radial movement of the stiffening layer 3 outward is executable, which presses the stiffening layer 3 onto the outer tube 4 and thus stiffens it. The choice of materials and the mode of operation are otherwise analogous to FIG. 1.

FIG. 10 shows a wire device with a torsionally rigid structure 9 formed in the outer tube 4.

The wire device 100 is shown in open sections of each layer in the profile for better visibility.

From the outside to the inside, the outer tube 4, the stiffening layer 3 and the pressure tube 2 are shown.

The outer tube 4 comprises a hypotube predominantly of metal, which has helical cuts 11 along the longitudinal axis of the wire device 100.

The outer tube 4 is designed to be essentially immobile radially.

The helical cuts 11 along the outer tube 4 form the torsionally rigid structure 9. In this embodiment, the helical cuts 11 are arranged along a direction of rotation about the longitudinal axis. The helical cuts 11 do not extend continuously along the surface of the outer tube 4, but have material bridges 12. In this embodiment, the helical cuts 11 each extend around the outer tube 4 by nearly one revolution and are arranged alternately with staggered helical cuts 11.

FIG. 11 shows an alternative design of the wire device 100 with a torsionally rigid structure 9 formed in the stiffening layer 3. The torsionally rigid structure 9 on the stiffening layer 3 is designed to be radially movable.

The choice of materials and mode of operation is otherwise analogous to FIG. 10 and no further description is given. FIG. 12 shows a wire device 100 with a tip 16 and a fluid coupling 8. The outer tube 4, the stiffening layer 3 and the pressure tube 2 of the stiffening section 101 have been partially removed for better illustration.

The tip 16 at the proximal end 15 in FIG. 12 terminates directly with the outer tube 4 and is shown tapered for clarity. This is only a perspective representation. The outer tube 4 is made hydrophilic by a coating 5 with polyvinylpyrrolidone. The fluid coupling 8 has a thread 13 for attaching a pressure device (not shown in FIG. 12).

The fluid coupling 8, in combination with the pressure device, allows fluid to be introduced to stiffen the stiffening section 101. The fluid coupling 8 at the distal end 30 includes a check valve (not shown in FIG. 12), so that the pressure device can be removed after pressure is applied without pressure loss and/or escape of fluid. The pressure tube 2 surrounds an inner space 1 into which the fluid can be introduced. To release pressure and fluid, the fluid coupling 8 has a release device on the check valve (not shown in FIG. 12). The release device is designed to be releasable in the blocking direction by means of a core which can be inserted into the fluid coupling, so that flow is possible in both directions. In this way, the pressure can be removed and the stiffening section 101 can be made flexibly movable again.

FIG. 13 shows a longitudinal section of the tip 16 of a wire device 100. The tip 16 is adjacent to the proximal end 15 in a pressure-tight manner with the pressure tube 2 and has a rounded shape 17. In this embodiment, the rounded shape 17 is formed of resilient polysiloxane. The tip has substantially the same cross-section as the stiffening layer 101 and the outer tube 4.

The stiffening layer 3 and the interior 1 are also formed along a stiffenable portion of the tip 16. The tip 16 has a region with a spiral reinforcement 23 and forms a continuation of the stiffening section 101 with another spiral reinforcement 19. The dashed black line illustrates the transition from the stiffening section 101 with other reinforcement 19 to the tip 16 with the weaker reinforcement 23. Iron dust continues to be added to the material in the region of the tip 16, so that movement can be visualized under X-ray methods.

The stiffening section of the tip 16 exhibits high elasticity because the winding of the reinforcement 23 has a greater spacing than the winding of the reinforcement 19. Thus, the stiffening section 101 toward the proximal end 15 exhibits increased elasticity, which facilitates bending of the wire device 100 in the vessels. The outer tube 4 has not been shown as a continuous surface in FIG. 13 for clarity, but is formed to be pressure-tight. The reinforcements 19, 23 are made of stainless steel.

FIG. 14 shows a longitudinal section of a wire device 100 having a tip 16 and stiffening section 101.

Repeated descriptions are omitted and reference is made to FIG. 13. The area of tip 16 with a different spiral reinforcement 23 and the rounded shape 17 has been marked A. In this context, varying elasticity in different areas of the tip 16 would also be conceivable. The area of the stiffening section 101 with other reinforcement 19 has been marked B.

The stiffening section 101 is arranged adjacent to an unstiffenable tubing apparatus 22. This unstiffenable tubing apparatus 22 has a continuation of the interior 1 and only a coaxial tubing layer 14. The coaxial hose layer 14 has a higher layer thickness and is tapered to the stiffening section 101 and formed pressure-tight with the pressure tube 2. The wire device 100 shown in FIG. 14 is designed to maintain torque along its entire length since it includes a torsionally rigid structure 9 (not shown in FIG. 14) formed along the entire longitudinal axis of the wire device 100.

FIG. 15 illustrates one embodiment of a push-on device 300 that is formable and guidable with two wire devices 100. The push-on device 300 has two separate lumens, each with an opening at a distal end 28 of the push-on device 300. A wire device 100 is insertable into each of these openings. In this embodiment, one lumen has an opening to the proximal end 27 of the push-on device 300 such that the wire device 100 can be used as a guide wire 24. The wire device 100 as a guide wire 24 can form a curvature 26, and is formed to be stiffenable in this curved orientation by the stiffening section 101 (not shown in FIG. 15). This curvature 26 can be used to specify a bend within the vessels. The push-on device 300 can be pushed along this predetermined curvature 26 and is movable along the curvature relative to the guide wire 24 in vessels.

The wire device 100 as shaped wire 25 is insertable into the other lumen. The other lumen is closed to the proximal end 27 of the push-on device 300, so that a shape can be given to the push-on device 300 in the region 6 of the proximal end 27 by the wire device 100 as shaped wire 25.

In this embodiment, the wire device 100 as a shaped wire 25 provides a shape only when the stiffening section 101 is stiffened in the region 6 of the proximal end 27.

1. A wire device comprising a stiffening section comprising a pressure tube, a stiffening layer and an outer tube, the stiffening layer being arranged concentrically around the pressure tube and the outer tube being arranged concentrically outside the stiffening layer the outer tube being radially rigid and the stiffening layer being radially movable, wherein a pressure can be built up in the pressure tube in such a way that the stiffening layer can be pressed from the inside against the outer tube and thus the stiffening section can be reversibly stiffened under pressure.

2. The wire device according to claim 1, wherein the pressure tube is expandable.

3. The wire device according to claim 1 wherein the stiffening layer comprises at least two stabilizing elements, the orientation of the stabilizing elements having at least a partial vector in the longitudinal direction of the wire device.

4. The wire device according to claim 3, wherein the stabilizing elements are made of at least one of a metal and a plastic.

5. The wire device according to claim 1, wherein the outer tube is designed to be pressure-tight and is made of at least one of a plastic and a metal.

6. The wire device according to claim 1, wherein the wire device comprises a torsionally rigid structure.

7. The wire device according to claim 1, wherein the wire device comprises a fluid coupling through which a fluid can be introduced into the pressure tube.

8. The wire device according to claim 1, wherein the wire device at least partially comprises an X-ray visible material.

9. The wire device according to claim 1, wherein a proximal end of the wire device comprises a tip which closes the proximal end of the wire device in a pressure-tight manner.

10. The wire device according to claim 1, wherein a hydrophilic or hydrophobic coating is arranged on the outside of the outer tube.

11. The wire device according to claim 1, wherein the stiffening section forms part or all of the wire device.

12. The wire device according to claim 1, wherein the outer tube has an outer diameter of at most 0.035 inch.

13. The wire device, according to claim 1, wherein the pressure tube and the stiffening layer are connected to each other.

14. A method for selectively stiffening a wire device according to claim 1, wherein by introducing a fluid into the pressure tube through the stiffening layer and the outer tube, the wire device is stiffened.

15. The method according to claim 14, wherein by reducing the pressure in the pressure tube, the wire device becomes movable again.

16. The wire device according to claim 1, wherein the wire device is a wire device for insertion into a body passage.

17. The wire device according to claim 1, wherein the pressure in the pressure tube can be relieved so that the stiffening section is flexible again.

18. The wire device according to claim 2, wherein the pressure tube is at least one of elastic and unfoldable.

19. The wire device according to claim 3, wherein the stabilizing elements are displaceable relative to one another.

20. The wire device according to claim 4, wherein a plurality of stabilizing elements is braided to form a braided structure.

21. The wire device according to claim 6, wherein the torsionally rigid structure is one of a hypotube, a helical structure, and a twisted structure.

22. The wire device according to claim 7, wherein a cross section through the fluid coupling has at most the same extension as a cross section of the stiffening section.
